(19) United States
(12) Patent Application Publication
(10) Pub. No.: US 2025/0221353 A1
(43) Pub. Date: Jul. 10, 2025

(54) DOUBLE-FLOWERED TORENIA PLANT WITH EXCELLENT ORNAMENTAL PROPERTIES, AND METHOD FOR PRODUCING SAME

(71) Applicant: NATIONAL AGRICULTURE AND FOOD RESEARCH ORGANIZATION, Tsukuba-shi, Ibaraki (JP)

(72) Inventor: Takaaki Nishijima, Tsukuba-shi, Ibaraki (JP)

(21) Appl. No.: 18/841,145

(22) PCT Filed: Feb. 20, 2023
(86) PCT No.: PCT/JP2023/005925

§ 371 Date: Aug. 23, 2024

(30) Foreign Application Priority Data
Mar. 11, 2022 (JP) 2022-037742

(51) Int. Cl. A01H 1/02; A01H 5/02; A01H 6/00

(52) U.S. Cl. CPC A01H 1/02; A01H 5/02; A01H 6/185; A01H 6/305

(57) ABSTRACT

A method for producing a double-flowered Torenia plant including the steps of obtaining an F1 population by crossing a Torenia plant belonging to a semi-double-flowered strain and a Torenia plant belonging to a multiple-carpel strain, and of selecting, from a second- or subsequent-generation hybrid population, a fully double-flowered individual having a flower with 15 or more developed petals, the second- or subsequent-generation hybrid population being a population of a second or subsequent generation derived from a first-generation hybrid population, and the first-generation hybrid population being the F1 population that has been obtained. A fully double-flowered Torenia plant that belongs to a fully double-flowered strain, a progeny population thereof, or their mutant population, and that has a flower with 15 or more developed petals.

### FIELD OF THE INVENTION

The present invention relates to highly ornamental Torenia plants bearing double flowers and methods for producing these Torenia plants.

### BACKGROUND OF THE INVENTION

Intense summer heat is becoming common due to global warming. As a result, major summer bedding plants such as petunias and marigolds are increasingly suffering from poor growth under high temperature, and demand for plants that are more tolerant to high temperature is growing. However, few heat-tolerant bedding plants are as highly ornamental as petunias and marigolds, and moreover, their usage as summer bedding or potted plants is limited due to various reasons.

Torenia has high environmental adaptability: it is extremely tolerant to high temperature compared with other flowering plants and can be grown under a wide range of light conditions from full sun to shade. Thus, Torenia is a popular flowering plant not only in Japan but also in the United States. However, it is difficult to obtain flower shape mutants of Torenia. Though Torenia has been cultivated in Japan since the Meiji Era (i.e., since the late 19^{th} century), variation in its flower shape has been poor, and there are only single- and small-flowered cultivars. If more gorgeous and attractive flowers, such as double flowers, can be obtained, Torenia is expected to become a major plant for flower beds and pots in summer, as it has excellent environmental adaptability.

As shown above, though Torenia has excellent heat resistance, its existing cultivars bear single flowers only, and production of highly ornamental double-flowered Torenia strains that can be utilized in designing flower beds in summer is desired.

However, it is difficult to obtain flower shape mutants of Torenia by conventional mutation methods such as heavy-ion beam irradiation and chemical mutagen treatment. Like other plant species, Torenia is reported to have floral homeotic genes, and a “Petaloid” mutant of Torenia, in which the stamens and the pistil are converted to petals by inhibition of the C-class gene function, is reported. Yet, a Torenia mutant produced by the reduction in C-class gene function has only nine developed petals (five original petals and four converted petals), and it should be admitted that this mutant does not bear sufficiently ornamental double flowers (Non-Patent Document 1). Meanwhile, Patent Document 1 reports a completely sterile Torenia plant obtained by almost fully suppressing the C-class gene function by genetic recombination. Yet, though the number of petals is increased, these petals are underdeveloped and do not expand fully. Thus, it should be admitted that the ornamental value of this Torenia flower is not enhanced (Patent Document 1).

Non-Patent Document 5 reports a technique to increase the number of petal lobes of Torenia flowers by applying forchlorfenuron (CPPU) to flower buds at specific floral stages. However, the morphological changes of the flowers achieved by this technique are temporal, as they are brought about by chemical treatment during flower bud formation (i.e., the genetic background of the plant is not modified in this technology). Accordingly, the morphological trait of these flowers is not genetically transmitted to subsequent generations. In addition, in this technique, double flowers can be obtained only when CPPU is applied to flower buds at specific floral stages. Other untreated flower buds do not develop into double flowers, and the Torenia plant does not continue to bear double flowers without the CPPU treatment.

As described above, it is difficult to obtain genetically altered flower shape mutants of Torenia. Under such technical circumstances, the inventor of the present invention obtained a strain named “flecked”, which is a mutable strain of Torenia with high transposition activity of the transposon, from mutants obtained by chemical mutagen treatment. Due to the mutation trait caused by the high transposition activity of the transposon in the “flecked” strain, screening of the self-pollinated progeny of the “flecked” strain enabled to generate many various Torenia mutants (Non-Patent Document 2). However, even by the selection from the selfed descendants of the highly mutable strain “flecked”, neither a mutant bearing highly ornamental double flowers nor a method to produce such a mutant could not be discovered.

### CITATION LIST

### Patent Document

Patent Document 1: JP2017-18100A

### Non-Patent Documents

Non-Patent Document 1: T. Nishijima et al., The Horticulture Journal, 2016, Volume 85, Issue 3, pp. 272-283. A Novel “Petaloid” Mutant of Torenia (Torenia fournieri Lind. ex Fourn.) Bears Double Flowers through Insertion of the DNA Transposon Ttf1 into a C-class Floral Homeotic Gene.
Non-Patent Document 2: T. Nishijima et al., Journal of the Japanese Society for Horticultural Science, 2013, Volume 82, Issue 1, pp. 39-50. A Torenia (Torenia fournieri Lind. ex Fourn.) Novel Mutant ‘Flecked’ Produces Variegated Flowers by Insertion of a DNA Transposon into an R2R3-MYB Gene.
Non-Patent Document 3: T. Niki et al., The Horticulture Journal, 2016, Volume 85, Issue 4, pp. 351-359. Conversion of Abaxial to Adaxial Petal in a Torenia (Torenia fournieri Lind. ex Fourn.) Mutant Appeared in Selfed Progeny of the Mutable Line “Flecked”.
Non-Patent Document 4: T. Nishijima et al., Horticultural Research (Japan), 2016, Volume 15 (Suppl.1), p. 203. A Torenia mutant exhibiting dark violet adaxial petal which appeared in the selfed progeny of a mutable line ‘Flecked’.
Non-Patent Document 5: T. Nishijima et al., Scientia Horticulturae, 2006, Volume 109, pp. 254-261. Change in flower morphology of Torenia fournieri Lind. induced by forchlorfenuron application.

### SUMMARY OF THE INVENTION

### Technical Problem

The present invention has been achieved to address the aforementioned problems of the conventional techniques, and it is an object thereof to provide a highly ornamental Torenia plant bearing double flowers.

### Solution to Problem

As a result of intensive studies conducted to solve the aforementioned problems, the inventor of the present invention found a semi-double-flowered mutant having a slightly increased number of petals through selection from the selfed descendants of the “flecked” strain described above. This semi-double-flowered mutation showed a single recessive trait. Though this semi-double-flowered Torenia had a slightly increased number of petals, the number of petals was as low as seven to ten, and it was hard to conclude that this Torenia was a highly ornamental, double-flowered plant.

Also, the present inventor found another mutant having an increased number of carpels constituting the pistil through selection from the selfed descendants of the “flecked” strain. This “multiple carpel” mutation having an increased number of carpels showed a single recessive trait.

The inventor crossed the semi-double-flowered strain and the multiple-carpel strain that were obtained as described above and investigated the flower shape of the individuals in the progeny generations. Then, surprisingly, highly ornamental, double-flowering Torenia plants having a sufficient number of developed petals (i.e., Torenia plants having a fully double-flowered trait) appeared at a certain frequency in the F2 generation of the cross. The frequency of this double-flowered mutant was consistent with the theoretical frequency of a double mutant, suggesting that this fully double-flowered trait was expressed as a result of the double mutation occurred by the combination of the semi-double-flowered mutation and the multiple-carpel mutation.

The multiple-carpel mutant, which is one of the parents of the cross, occurs by pistil mutation, and the crossing experiment of the present disclosure has proved for the first time ever that production of a double mutant through the cross between the multiple-carpel mutant and the semi-double-flowered mutant enables to realize a remarkably increased number of sufficiently developed petals.

Moreover, in addition to the mutants described above, the present inventor found a mutant bearing bilaterally and dorsoventrally symmetric flowers resembling begonia flowers from the selfed progeny of the “flecked” strain. While a normal-type Torenia flower is only bilaterally symmetric, the flower of this begonia-like mutant has dorsoventral symmetry (symmetric about a horizontal line) in addition to bilateral symmetry. This begonia-like mutation showed a single recessive trait.

The inventor further crossed the fully double-flowered strain and one of the strains bearing begonia-like flowers that were obtained as described above. Then, surprisingly, from the F2 generation derived from the cross, highly ornamental, double-flowered Torenia plants which had the flower shape characteristics of a fully double-flowered plant, having flowers with a sufficient number of developed petals, and moreover, whose petals were arranged radially around the floral axis to form a double flower with a substantially radially symmetric corolla (i.e., Torenia plants having flower shape characteristics of a “carnation-like fully double-flowered strain”), were obtained at a certain frequency. The frequency of the plants having these characteristics was consistent with the theoretical frequency of a multiple mutant obtained by the combination of each mutation, suggesting that this carnation-like, fully double-flowered trait was expressed as a result of the multiple mutation occurred by the combination of the double mutation of the fully double-flowered Torenia and the mutation of the begonia-like Torenia.

Here, the mutations possessed by the fully double-flowered strain and the begonia-like strain, which are the parents of the cross, do not cause the corolla of these two strains to be substantially radially symmetric. The crossing experiment of the present disclosure has proved for the first time that a phenotype, in which the flower has a large number of petals being arranged randomly and radially around the floral axis to form a double flower and the entire corolla is substantially radially symmetric, is expressed in the multiple mutant produced by the cross between the fully double-flowered strain and the begonia-like strain.

The present invention was arrived at based on the above-mentioned findings and specifically relates to aspects of the invention described below.
[1] A method for producing a double-flowered Torenia plant, the method including the steps of:
obtaining an F1 population by crossing
   (Parent 1)
   a Torenia plant belonging to a semi-double-flowered strain (FERM BP-22419) or
   a Torenia plant that belongs to a progeny population or a mutant population of the Torenia plant belonging to the semi-double-flowered strain (FERM BP-22419) and has a flower with 7 to 10 developed petals; and
   (Parent 2)
   a Torenia plant belonging to a multiple-carpel strain (FERM BP-22420) or
   a Torenia plant that belongs to a progeny population or a mutant population of the Torenia plant belonging to the multiple-carpel strain (FERM BP-22420) and has a flower with an increased number of carpels; and selecting, from a second- or subsequent-generation hybrid population, an individual that has a flower with 15 or more developed petals, the second- or subsequent-generation hybrid population being a population of a second or subsequent generation derived from a first-generation hybrid population, and the first-generation hybrid population being the F1 population that has been obtained.
[2] A method for producing a double-flowered Torenia plant, the method including the steps of:
obtaining an F1 population by crossing
   (Parent 3)
   the double-flowered Torenia plant obtained by the method according to aspect 1,
   a Torenia plant belonging to a fully double-flowered strain (FERM BP-22437), or
   a Torenia plant that belongs to a progeny population or a mutant population of the double-flowered Torenia plant obtained by the method according to aspect 1 or of the Torenia plant belonging to the fully double-flowered strain (FERM BP-22437) and that has a flower with 15 or more developed petals; and
   (Parent 4)
   a freely selected Torenia plant; and selecting, from a second- or subsequent-generation hybrid population, an individual that has a flower with 15 or more developed petals and has a desired trait, the second- or subsequent-generation hybrid population being a population of a second or subsequent generation derived from a first-generation hybrid population, and the first-generation hybrid population being the F1 population that has been obtained.
[3] A method for producing a double-flowered Torenia plant, the method including the steps of:
obtaining an F1 population by crossing
   (Parent 3′)
   the double-flowered Torenia plant obtained by the method according to aspect 1,
   a Torenia plant belonging to a fully double-flowered strain (FERM BP-22437), or
   a Torenia plant that belongs to a progeny population or a mutant population of the double-flowered Torenia plant obtained by the method according to aspect 1 or of the Torenia plant belonging to the fully double-flowered strain (FERM BP-22437) and that has a flower with 15 or more developed petals; and
   (Parent 5)
   a Torenia plant belonging to a begonia-like strain (FERM BP-22422) or
   a Torenia plant that belongs to a progeny population or a mutant population of the Torenia plant belonging to the begonia-like strain (FERM BP-22422) and that has a flower whose shape is bilaterally and dorsoventrally symmetric; and
selecting, from a second- or subsequent-generation hybrid population, an individual that has a flower with 15 or more developed petals arranged radially around a floral axis to form a double flower with a substantially radially symmetric corolla, the second- or subsequent-generation hybrid population being a population of a second or subsequent generation derived from a first-generation hybrid population, and the first-generation hybrid population being the F1 population that has been obtained.
[4] A method for producing a double-flowered Torenia plant, the method including the steps of:
obtaining an F1 population by crossing
   (Parent 6)
   the double-flowered Torenia plant obtained by the method according to aspect 3,
   a Torenia plant belonging to a carnation-like fully double-flowered strain (FERM BP-22418), or
   a Torenia plant that belongs to a progeny population or a mutant population of the double-flowered Torenia plant obtained by the method according to aspect 3 or of the Torenia plant belonging to the carnation-like fully double-flowered strain (FERM BP-22418) and that has a flower with 15 or more developed petals arranged radially around a floral axis to form a double flower with a substantially radially symmetric corolla; and
   (Parent 7)
   a freely selected Torenia plant; and
selecting, from a second- or subsequent-generation hybrid population, an individual that has a flower with 15 or more developed petals arranged radially around a floral axis to form a double flower with a substantially radially symmetric corolla and that has a desired trait, the second- or subsequent-generation hybrid population being a population of a second or subsequent generation derived from a first-generation hybrid population, and the first-generation hybrid population being the F1 population that has been obtained.
[5] A method for producing a double-flowered Torenia plant,
wherein the double-flowered Torenia plant obtained by the method according to any one of aspects 1 to 4 is used as one parent or both parents of a cross.
[6] A double-flowered Torenia plant,
wherein the double-flowered Torenia plant is any one of the following (Plant 1), (Plant 2), or (Plant 3):
   (Plant 1)
   a Torenia plant belonging to a fully double-flowered strain (FERM BP-22437) or a carnation-like fully double-flowered strain (FERM BP-22418),
   (Plant 2)
   a Torenia plant that belongs to a progeny population of the Torenia plant described in (Plant 1) and that has a flower with 15 or more developed petals; or
   (Plant 3)
   a Torenia plant that belongs to a mutant population of the Torenia plant described in (Plant 1) or (Plant 2) and that has a flower with 15 or more developed petals.
[7] A double-flowered Torenia plant,
wherein the double-flowered Torenia plant is any one of the following (Plant 1′), (Plant 2′), or (Plant 3′):
   (Plant 1′)
   a Torenia plant belonging to a carnation-like fully double-flowered strain (FERM BP-22418),
   (Plant 2′)
   a Torenia plant that belongs to a progeny population of the Torenia plant described in (Plant 1′) and that has a flower with 15 or more developed petals arranged radially around a floral axis to form a double flower with a substantially radially symmetric corolla; or
   (Plant 3′)
   a Torenia plant that belongs to a mutant population of the Torenia plant described in (Plant 1′) or (Plant 2′) and that has a flower with 15 or more developed petals arranged radially around a floral axis to form a double flower with a substantially radially symmetric corolla.
[8] A method for producing a double-flowered Torenia plant, wherein the double-flowered Torenia plant according to aspect 6 or 7 is used as one parent or both parents of a cross.
[9] A plant body of the double-flowered Torenia plant according to aspect 6or 7.
[10] A method for cultivating a double-flowered Torenia plant, a method for producing a plant body of a double-flowered Torenia plant, or a method for producing a strain of a double-flowered Torenia plant,
wherein the double-flowered Torenia plant according to aspect 6 or 7 is used.

### Advantageous Effects of the Invention

The present invention enables to provide highly ornamental, double-flowered Torenia plants. Also, the present invention enables to provide parental lines that can be used to produce highly ornamental, double-flowered Torenia plants.

### BRIEF DESCRIPTION OF DRAWINGS

FIG. 1 shows the photographic images of Torenia flowers. (Left) Normal type. (Right) “Flecked” strain.

FIG. 2 shows the photographic images of flowers of parental lines used to produce a Torenia plant having a fully double-flowered trait and a flower of a double-flowered strain produced. (Top left) Semi-double-flowered strain. (Top right) Multiple-carpel strain. (Bottom) Fully double-flowered strain.

FIG. 3 shows the photographic images of flowers of parental lines used to produce a Torenia plant having a carnation-like fully double-flowered trait. (Top left) Begonia-like strain. (Top right) Strain having uniformly pigmented adaxial and abaxial petals. (Bottom) Begonia-like strain (uniformly pigmented type).

FIG. 4 shows the photographic images of flowers of a carnation-like fully double-flowered strain produced. (Left) Photographic image of the flower and the surrounding plant parts. (Right) Photographic image of the flower without the surrounding plant parts.

FIG. 5 is a schematic view of a breeding process conducted to produce the double-flowered Torenia plants in the Examples of the present disclosure.

### DETAILED DESCRIPTION OF THE EMBODIMENTS

Hereinafter, embodiments of the present invention will be described in detail, but the technical scope of the present invention is not limited to the embodiments having all of the following features. Also, embodiments including features other than those described below are not excluded from the technical scope of the present invention.

The present application claims priority to Japanese Patent Application Serial No. 2022-037742 filed with the Japan Patent Office by the applicant of the present invention, the entire contents of which are incorporated herein by reference.

### 1. Terminology

Torenia plants according to the present disclosure include (a) plants that belong to the species Torenia fournieri Lind. ex Fourn. of the genus Torenia, Linderniaceae. Specifically, the Torenia plants according to the present disclosure include plants of various strains (including cultivars) and populations of Torenia fournieri. In addition to artificially produced cultivars, strains and populations, the Torenia plants according to the present disclosure also include wild varieties and local populations.

Moreover, the Torenia plants according to the present disclosure include hybrids (including plants belonging to a hybrid population) that are produced by a cross between Torenia plants each belonging to a strain (including cultivar) or a population of the species Torenia fournieri. They also include plants belonging to an offspring population of these hybrids. They further include plants that belong to a hybrid population produced by a cross between strains, between populations, or between a strain and a population. They also include plants that belong to an offspring population of the plants belonging to the hybrid population.

The Torenia plants according to the present disclosure include (b) hybrids (including plants belonging to a hybrid population) produced by a cross between a plant that belongs to the species fournieri and a plant that belongs to the genus Torenia but does not belong to the species fournieri, and moreover, plants belonging to an offspring population of these hybrids. That is, the Torenia plants according to the present disclosure include hybrids produced by the cross between the species fournieri and another species of the genus Torenia, and moreover, the Torenia plants according to the present disclosure include offsprings of these hybrids.

An example of the “plant that belongs to the genus Torenia but does not belong to the species fournieri,” which serves as one of the parents of the hybrid produced, can be a plant belonging to a species, a strain (including a cultivar) or a population that is classified as the genus Torenia and that can produce a hybrid by crossing with the species fournieri.

Examples of the “plant that belongs to the genus Torenia but does not belong to the species fournieri” include the species Torenia concolor, the species Torenia baillonii, and the species Torenia bicolor, which are close relatives of Torenia fournieri. They also include plants that belong to strains (including cultivars) or populations of these close relatives, hybrids (including hybrid populations) produced by a cross between these strains (including cultivars) or populations, and offspring populations of these hybrids (including hybrid populations). Here, Torenia concolor, Torenia baillonii and Torenia bicolor are close relatives of Torenia fournieri that are confirmed to produce hybrids by crossing with Torenia fournieri. Also, plants other than the three close relatives mentioned above can be the “plants that belong to the genus Torenia but do not belong to the species fournieri” if hybrids can be produced by crossing with the species Torenia fournieri.

The Torenia plants according to the present disclosure also include (c) plants that belong to a hybrid population produced by the cross between Torenia plants described in (b) above (i.e., hybrids or their offsprings produced by the cross between the plant that belongs to the species fournieri and the plant that belongs to the genus Torenia but does not belong to the species fournieri), and moreover, plants that belong to an offspring population of these plants belonging to the hybrid population.

The “plants that belong to the genus Torenia but do not belong to the species fournieri” can also include the Torenia plants described in (b) and (c) above. These Torenia plants are preferable examples of the “plants that belong to the genus Torenia but do not belong to the species fournieri”, as they are hybrids or their offsprings having a strong genetic background inherited from the species fournieri.

Also, the “plants that belong to the genus Torenia but do not belong to the species fournieri” can include the hybrids (hybrid populations) produced by crossing between a Torenia plant described in (b) or (c) above and a plant belonging to the species fournieri, and moreover, the offspring populations of these hybrids. Similarly, in their subsequent generations, the hybrids (hybrid populations) produced by crossing with the species fournieri and the offspring populations of these hybrids can also be the examples of the “plants that belong to the genus Torenia but do not belong to the species fournieri”.

In certain embodiments, in the production of hybrid populations (hybrids) by the cross described in the paragraphs above (including (a), (b) and (c) mentioned above), one or more than one hybrid populations are produced by one or more than one crossings. That is, in some such embodiments, the hybridization includes the production of two or more hybrid populations by changing the combination of parents that are described in the paragraphs above. Meanwhile, the production of the offspring populations of the hybrid populations includes the production of offspring populations by the cross between two or more different hybrid populations. The hybrid populations and their offspring populations also include plants that belong to the hybrid populations obtained by the cross between strains, between populations, or between a strain and a population, and plants that belong to the offspring populations of these hybrid populations.

The “offspring populations” of the hybrid or the like described in the paragraphs above (including (a), (b) and (c) above) refer to the populations of the second and subsequent generations following the hybrid population (hybrid: the first-generation hybrid population) produced by a cross. Also, the explanation about “the first-generation hybrid population” and “the subsequent-generation hybrid population derived from the first-generation hybrid population” described in Section 1 (this section), paragraphs below, and in Section 3 below can be adopted to explain the offspring population and the progeny population.

A flower of a normal-type (wild-type) Torenia plant is single-flowered and bilaterally symmetric and has five petals arranged around the floral axis. The basal parts of the petals are fused to form a gamopetalous flower.

The abaxial and lateral petals of a normal-type Torenia flower have similar shapes, while the shape of the adaxial petals is different from that of the abaxial and lateral petals.

The abaxial and lateral petals have similar spatulate shapes. The word “spatulate” is a scientific term that describes the shape of a leaf or a petal, and a spatulate shape is generally as follows: the width is narrow and becomes gradually wider from the basal part toward the lower middle part, the width becomes much wider from the lower middle part toward the middle part, the width increases a little from the middle part toward the upper middle part, reaching the maximum width at the upper middle part, and the width narrows from the upper middle part toward the upper end, forming a round shape at the upper end of the petal. This shape resembles a rice spoon, if it is compared to an object.

The two spatulate adaxial petals are completely fused to form a wide and large spatulate shape.

While Torenia has various flower colors, the pigmentation pattern tend to vary depending on the position of the petal in the flower. The adaxial petals tend to be pigmented in paler colors than the abaxial and lateral petals. The basal parts of the abaxial and lateral petals tend to be pigmented in pale colors, while the upper parts of the abaxial and lateral petals tend to be pigmented in dark colors. The abaxial petal often has a yellow nectar guide in its central region.

The term “flecked” as used herein refers to a strain of Torenia which was produced by the present inventor and other researchers as a population having high transposition activity of the transposon. Due to its special mutation trait caused by the active transposon, the “flecked” strain enables to obtain many various Torenia mutants through screening of its selfed descendants (Non-Patent Document 2).

The term “progeny population” as used herein refers to a filial population (filial population obtained by crossing or selfing) and its offspring population whose one parent is a specifically selected Torenia plant or whose both parents are specifically selected Torenia plants. It also includes a hybrid population (filial population) and its offspring population produced by a cross between different cultivars or strains.

Here, offspring populations include a population obtained from a cross between different filial populations produced, a population obtained from a cross between individuals in the same filial population produced, and a population obtained from selfing of an individual in a filial population produced. Moreover, subsequent generations obtained from selfing of an individual in the offspring population, crossing between individuals in the same offspring population, and crossing between individuals belonging to different offspring populations are also regarded as offspring populations.

In other words, offspring populations include a population obtained from a cross between offspring populations produced, a population obtained from a cross between a filial population and an offspring population produced, a population obtained from a cross between individuals within the same offspring population produced, and a population obtained from selfing of an individual belonging to an offspring population produced.

The progeny populations of these populations are also included in the term “progeny population”.

The term “mutant population (variant population, mutant strain)” as used herein refers to a population in which a mutant trait of a mutant (mutant individual) produced by mutation, transgenesis, genome editing, and the like It also includes progeny populations having the can be inherited stably. mutant trait.

The term “first-generation hybrid population” as used herein refers to the F1 population (hybrid population) produced by the cross (hybridization) between different populations or different cultivars/strains/varieties. In certain embodiments, the “hybridization” of the present disclosure includes the production of one or more than one F1 populations by one or more than one crossings between parents described in the paragraphs of the present disclosure. In some such embodiments, the hybridization includes the production of two or more F1 populations by changing the combination of said parents. Meanwhile, the production of the offspring population of the hybrid population includes the production of the offspring population by the cross between two or more different F1 populations (between different populations).

The term “second-generation hybrid population” as used herein refers to a population of the second generation produced by a cross within the same F1 population, a cross between different F1 populations, or a selfing of an individual that belongs to an F1 population.

As used herein, a second-generation hybrid population produced by a cross within the same F1 population or a selfing of an individual that belongs to an F1 population is regarded as an F2 population. Similarly, a third-generation hybrid population produced by a cross within the same F2 population or a selfing of an individual that belongs to an F2 population is regarded as an F3 population. Also, a fourth-generation hybrid population produced by a cross within the same F3 population or a selfing of an individual that belongs to an F3 population is regarded as an F4 population.

In other words, an nth-generation hybrid population produced by a cross within the same Fn-1 population or a selfing of an individual that belongs to an Fn-1 population is regarded as an Fn population (n: a natural number indicating the generation).

As used herein, the term “a cross within the same population” refers to a cross between individuals that belong to the same population. The term “a cross between populations” refers to a cross between individuals that belong to two different populations. The cross includes random mating between individuals. The term “selfing” refers to reproduction by self-pollination of an individual.

As used herein, terms indicating “a cross between two populations” can be regarded as being equivalent to “hybridization”.

An offspring population of a certain population mentioned in this specification refers to the second- or subsequent-generation hybrid population that follows the hybrid population (hybrid: first-generation hybrid population) generated by a cross, when hybridization (crossing) is involved.

In a wider definition, an offspring population of a certain population can indicate a population of a subsequent generation derived from a filial population (first-generation population) of a specific population (or a strain). More specifically, an offspring population of a certain population can be explained as a second- or subsequent-generation hybrid population derived from a filial population (first-generation population) of a specific population (or a strain) (i.e., an offspring population of a certain population can be a population produced by a cross within a population of a preceding generation, a cross between populations of a preceding generation or generations, or a selfing of an individual that belongs to a population of a preceding generation).

The explanation about “the first-generation hybrid population” and “the subsequent-generation hybrid population derived from the first-generation hybrid population” described in the paragraphs above and in Section 3 below can be adopted as the explanation of the offspring population and the progeny population.

The term “flower shape” as used herein indicates the shape of a flower. The term “flower color” as used herein indicates the pigmentation of a flower. The “front face” of a Torenia flower tends to face the horizontal direction (the front face of the flower tends to be perpendicular to the ground), with the adaxial side being the upper side and the abaxial side being the lower side. When the flower shape is explained in the present disclosure, the “adaxial side” of a branched flower indicates the side facing the stem of a plant body and is the upper side of the flower. The “abaxial side” of a branched flower indicates the side facing away from the stem of a plant body and is the lower side of the flower. The “bilateral” direction indicates the direction perpendicular to the dorsoventral direction of the front face of the flower.

### 2. Fully Double-Flowered Torenia Plant

One aspect of the present disclosure relates to a double-flowered Torenia plant which has a fully double-flowered trait, having a sufficiently large number of developed petals.

### [Flower Shape Trait (Morphological Trait of the Flower)]

### (Fully Double-Flowered Trait)

As used herein, a Torenia plant having the fully double-flowered trait refers to a highly ornamental, double-flowered Torenia plant whose flower has a sufficiently increased number of developed petals, with the petals arranged radially around the floral axis.

As to the flower shape trait of the fully double-flowered Torenia plant, if the Torenia flower has 15 or more developed petals, the flower shape can be considered as being double flowered and having high ornamental value. The preferable number of developed petals in a flower having the fully double-flowered trait can be 19 or more, 20 or more, 25 or more, 30 or more, or 33 or more, for example. The maximum number of the petals is not limited if the flower shape is ornamental, and it can be 80 or less, 60 or less, or 50 or less, for example.

In some preferable embodiments, the number of developed petals can range from 15 to 80, preferably 19 to 60, and more preferably 33 to 50. In the present specification, even if two or more petals are fused and seem to form one petal, the number of the original petals before being fused is regarded as the number of the petals of the flower (e.g., if two petals are fused and seem to form one petal, the number of the petals is counted as two). For example, in a normal-type Torenia plant, two adaxial petals are fused to form a large spatulate petal, and the fused petal is counted as two petals.

When the number of petals of a Torenia flower falls within the range described above, the petals are arranged densely (i.e., the gaps between petals are narrow) and radially around the floral axis. As a result, the flower can have a beautiful, double-flowered shape and can be regarded as being fully double-flowered. In contrast, when the number of the petals is less than the numbers described above (especially when it is less than 15), the petals arranged around the floral axis become sparse (i.e., the gaps between the petals are wide). Such a flower shape is not preferable and cannot be regarded to be fully double-flowered.

In order to achieve high ornamental properties of the flower shape, it is preferable that the petals of the fully double-flowered Torenia plant reach a sufficiently large size when they have developed (expanded and extended). Regarding the size of the petal in a preferable embodiment of the present disclosure, the petal length from the lower end to the upper end of the petal (the length from the basal part, which includes the fused tubular part of the gamopetalous flower, to the tip of the petal) is 20 mm or more, while the outer petals of a flower tend to be large and the inner petals tend to be small. The length (the length without the tubular part) of a lobe portion (a lobe that extends from the basal part which includes the fused tubular part of the gamopetalous flower) is preferably 5 mm or more. Meanwhile, the width of a petal (the width at the widest part of a petal) is preferably 5 mm or more. The maximum values of these lengths and width are not particularly limited, because the fully double-flowered Torenia plants include those bearing flowers with large petals. For example, the maximum values (in mm) can fall within the values shown in the Examples section below.

The flower of a fully double-flowered Torenia plant has a gamopetalous corolla whose petals are fused at their base and split repeatedly and intricately toward the upper end of the petal, and each lobed portion corresponds to a petal. Therefore, the number of petals of a Torenia flower can be counted as the number of lobes of a flower (e.g., “15 lobes or more”). In some cases, some of the petals are free from other petals, divided from them at the basal part like in a polypetalous flower. In one embodiment, the shape of a petal is equivalent to that of a normal-type Torenia plant (spatulate shape with a smooth margin).

In certain embodiments, the petal shape of the Torenia flower is mutated. When explained using scientific terms describing petal or leaf shapes, the shape of an entire petal can be linear, broad linear, needle-shaped, lanceolate, elliptic, oblong, filiform, ovate, obovate, cordate, obcordate, rhomboid, rhombus-oval, rounded, oblate, reniform, or the like. The shape of the upper end of a petal can be acuminate, acute, obtuse, rounded, emarginate, apiculate, round-apiculate, caudate, or the like. The shape of the basal part of a petal can be attenuate, cuneate, truncate, cordate, auriculate, sagittate, hastate, or the like. The shape of a petal margin can be entire, undulate, undulate-serrate, serrate, dentate, doubly serrate, incised, or the like.

Further, in some embodiments, a petal has a fan shape, a shape having projections along the margin, a shape having a divided margin, a shape with a round margin, an entirely round shape, a shape with a pointed margin, an entirely pointed shape, a shape having a margin indented at the center, a shape having a margin indented at both sides of the petal, a shape with a margin divided into many narrow segments, a shape entirely curled to the abaxial side, a shape in which the end of the margin is curled to the abaxial side, a shape entirely curled to the adaxial side, a shape in which the end of the margin is curled to the adaxial side, a frilled shape, a pleated shape, or the like.

In one embodiment, a petal has some of these characteristics.

As to the flower shape of the fully double-flowered Torenia plant, when viewed facing the front face of the flower, the petals described above are arranged radially around the floral axis and form a double flower. More specifically, the petals are arranged around the floral axis, spreading toward different directions, and the petal surfaces, which are adaxial to the floral axis, face the floral axis at different distances. As a result, the flower has many layers of petals, achieving a double-flowered shape.

In an embodiment of the flower shape of the fully double-flowered Torenia of the present disclosure, petals whose shapes are similar to each other are arranged radially. In another embodiment, petals of two or more different shapes (preferably, petals of two different shapes) are arranged radially.

In one embodiment of the flower shape having the fully double-flowered trait according to the present disclosure, the double flower has petals identical to those of the normal-type Torenia flower. Here, the normal-type Torenia flower has two different types of spatulate petals. More specifically, while abaxial and lateral petals have the same spatulate shapes, adaxial petals having spatulate shapes are fused longitudinally, forming a large spatulate petal.

In this embodiment, petals having the same shape as that of the adaxial petal of the normal-type Torenia flower are arranged on the adaxial side of the flower, petals having the same shape as that of the abaxial petal of the normal-type Torenia flower are arranged on the abaxial side of the flower, and petals having the same shape as that of the lateral petal of the normal-type Torenia flower are arranged on the lateral sides of the flower. As a result, in the fully double-flowered Torenia flower whose petal shapes are similar to those of the normal-type Torenia flower, the shape of the entire flower is bilaterally symmetric, layers of many developed, frilled petals are arranged surrounding the floral axis, and the flower can achieve a beautiful double-flowered shape.

More specifically, an example of the fully double-flowered Torenia plant having such a flower shape is a Torenia plant belonging to the “11118 strain” (FERM BP-22437).

In certain embodiments of the flower shape having the fully double-flowered trait according to the present disclosure, the double flower has mutant petals. Double flowers with various mutant petals are also included in these embodiments.

### (Flower Shape Characteristics of the Carnation-Like Fully Double-Flowered Torenia Plant)

In a preferable embodiment of the flower shape having the fully double-flowered trait of the present disclosure, when viewed facing the front face of the flower, the petals are arranged radially around the floral axis to form a double flower, and the entire corolla is “substantially radially symmetric”. Such characteristics are favorable in enhancing the ornamental value of the double-flowered Torenia plant. In this embodiment, the flowers are beautifully double-flowered, as the corolla is substantially radially symmetric when viewed facing the front face of the flower and layers of many mature petals are arranged surrounding the floral axis.

In some examples of the double-flowered Torenia plant that has such a flower shape and that is preferable from an ornamental point of view, petals of the same or similar shape or petals of two or more different shapes are randomly and radially arranged around the floral axis to form a substantially radially symmetric corolla. Since layers of many developed petals are arranged radially surrounding the floral axis, beautiful double flowers having frilled petals can be obtained. Since the double flower has frilled petals and a substantially radially symmetric corolla, its flower shape closely resembles that of a carnation flower. Therefore, in the present specification, this Torenia plant is referred to as the “carnation-like fully double-flowered” Torenia plant.

With respect to the flower shape in an example of this embodiment, petals of two or more (preferably two) different shapes are randomly and radially arranged to form a substantially radially symmetric corolla. In another example of this embodiment, petals of the same or similar shape are radially arranged to form a substantially radially symmetric corolla.

The term “substantially radially symmetric corolla” as used herein is different from the scientific term “actinomorphic corolla”. The term “substantially radially symmetric corolla” can be explained as a flower shape which is approximately symmetric about any radial axis passing through the floral axis when viewed facing the front face of the flower. It can also be explained as a flower shape in which the entire shape can be regarded as symmetric about any radial axis, though the shape may not be completely symmetric due to the random radial arrangement of the petals, the frilled shapes of the petals, or other reasons. It can also be explained as a flower shape in which any parts of the flower extending laterally to both sides from any radial axis passing through the floral axis of the flower have approximately the same shape except that they may not be completely identical due to the random radial arrangement of the petals, the frilled shapes of the petals, or other reasons.

In one such embodiment, the flower has two different types of frilled spatulate petals just like the fully double-flowered strain described above, and the petals are arranged radially and randomly around the floral axis to form a substantially radially symmetric corolla. More specifically, an example of the carnation-like fully double-flowered Torenia plant (one of the embodiments of the fully double-flowered Torenia plant) is the Torenia plant belonging to the “10868 strain” (FERM BP-22418).

In another embodiment, petals of the same or similar shape are arranged radially to form a substantially radially symmetric corolla.

### [Flower Color Characteristics (Pigmentation)]

The fully double-flowered Torenia plant according to the present disclosure has the beautiful flower shape having double-flowering characteristics as described above. Meanwhile, flower color characteristics regarding the pigmentation of the petals are not particularly limited, and the Torenia plants according to the present disclosure can include any Torenia plants with any pigmentation patterns if the characteristics related to the fully double-flowered shape are achieved.

The examples of the flower color characteristics are petals pigmented in the same way as those of the normal-type Torenia, petals pigmented with a single color, petals pigmented with dark and pale colors, petals having a certain pattern, and the like. The examples also include petals whose basal part is pigmented in a pale color and whose upper part is pigmented in a dark color, petals whose basal part and upper part are pigmented in different colors, petals with a picotee pattern, and the like.

It is especially preferable from an ornamental point of view that the radially arranged petals of the fully double-flowered Torenia plant according to the present disclosure are pigmented in a similar way (are not pigmented in various ways) or are pigmented in the same way, as the beauty of the flower shape of the fully double-flowered Torenia is greatly enhanced.

In a concrete example of the fully double-flowered Torenia plant having flowers with such pigmentation characteristics according to the present disclosure, the nectar guide or colored spot which usually appears on some of the petals of the normal-type Torenia flower disappears, each petal is pigmented with the same color, and as a result, the entire flower is pigmented with a single color. For example, the flower can be pigmented with a single color, such as violet, pale violet, dark violet, red violet, blue violet, pink, red, yellow, white, or the like. In another example of the fully double-flowered Torenia plant according to the present disclosure, the petals that are arranged radially have the same or similar appearances, as they are, for example, pigmented in a specific pattern or pigmented in the same or similar way with a dark color, a pale color, or dark and pale colors, and, as a result, the flowers are pigmented uniformly (not in various ways).

In yet another example of the fully double-flowered Torenia plant according to the present disclosure, two or more types of petals whose colors are different from each other yet each of which is pigmented with a single color are arranged radially and randomly to form a substantially radially symmetric corolla. In still another example, petals, which have different patterns or are pigmented in different ways in a dark color, a pale color, or dark and pale colors, are arranged radially and randomly, forming a substantially radially symmetric corolla. In still yet another example, petals, each of which has two or more colors, are arranged radially and randomly, forming a substantially radially symmetric corolla.

Similarly to the explanation about the flower shape and the corolla described above, the term “substantially radially symmetric” as used herein can be explained as the pigmentation pattern which is approximately symmetric about any radial axis passing through the floral axis when viewed facing the front face of the flower. The explanation about the flower shape and the corolla described above can be applied to the explanation about the “substantially radially symmetric” pigmentation, if the terms used for describing flower shapes or corollas are substituted to the terms used for describing pigmentation.

### [Other Characteristics]

The double-flowered Torenia plants according to the present disclosure encompass any Torenia plants that have desired favorable characteristics in addition to the characteristics related to the fully double-flowered shape as described above, if the aforementioned characteristics related to the fully double-flowered shape are satisfied.

The examples of such favorable characteristics are characteristics related to further improvement of the flower shape (e.g., characteristics contributing to achieving a larger corolla, a larger flower, a larger petal, improved flower color, improved pigmentation pattern, and the like), improvement of ornamental value of the entire plant body (e.g., characteristics contributing to increasing the number of flowers per plant, to improving the appearance of the entire plant under cultivation, to improving the leaf shape, and the like), characteristics related to growth (e.g., characteristics related to growth period length, plant hormone synthesis, and the like), characteristics related to tolerance to the environment (e.g., cold resistance, heat resistance, drought resistance, and the like), characteristics related to disease resistance, and characteristics related to reproduction (e.g., flowering control, self-incompatibility, cytoplasmic male sterility, and the like). The examples can also include other favorable characteristics.

### [Embodiments of the Double-Flowered Torenia Plant]

The double-flowered Torenia plants according to the present disclosure also include any Torenia plants having characteristics related to the flower color and/or other traits if they have the fully double-flowered trait (characteristics related to the morphology of the flower).

That is, the double-flowered Torenia plants according to the present disclosure include the Torenia plants that have flower shape characteristics related to the fully double-flowered trait described above. Also, the double-flowered Torenia plants of the present disclosure include the fully double-flowered Torenia plants obtained by the methods for producing the fully double-flowered Torenia plants as described below. An exemplary embodiment is the Torenia plant belonging to the “11118 strain” (FERM BP-22437), which is the fully double-flowered Torenia plant produced in the Examples section below.

Also, the fully double-flowered Torenia plants according to the present disclosure include any Torenia plants which belong to the progeny population of the fully double-flowered Torenia plants described above and whose flower shape has the fully double-flowered trait. Moreover, the fully double-flowered Torenia plants according to the present disclosure include any Torenia plants which belong to the mutant population of the fully double-flowered Torenia plants described above or the mutant population of the progeny population of the fully double-flowered Torenia plants described above and whose flower shape has the fully double-flowered trait.

An example of such Torenia plants belonging to the progeny population or the mutant population is a Torenia plant that belongs to the progeny population or the mutant population of the Torenia plant described above and that has flower shape characteristics (flower shape characteristics related to the fully double-flowered trait) marked by a sufficiently large number of developed petals (examples of the number of the petals are described above).

Moreover, the double-flowered Torenia plants according to the present disclosure include any Torenia plants that have the carnation-like fully double-flowered trait described above. An exemplary embodiment is the Torenia plant belonging to the “10868 strain” (FERM BP-22418), which is the carnation-like fully double-flowered Torenia plant produced in the Examples section below.

Also, the carnation-like fully double-flowered Torenia plant according to the present disclosure includes any Torenia plants which belong to the progeny population of the carnation-like fully double-flowered Torenia plant and whose flower shape has the carnation-like fully double-flowered trait. Moreover, the carnation-like fully double-flowered Torenia plant according to the present disclosure includes any Torenia plants which belong to the mutant population of the carnation-like fully double-flowered Torenia plant or the mutant population of the progeny population of the carnation-like fully double-flowered Torenia plant and whose flower shape has the carnation-like fully double-flowered trait.

An example of such Torenia plants belonging to the progeny population or the mutant population is the Torenia plant that belongs to the progeny population or the mutant population of the Torenia plant described above, that has the fully double-flowered trait marked by a sufficiently large number of developed petals (examples of the number of petals are described above), and that has flower shape characteristics in which the petals are arranged radially around the floral axis and forms a double flower and the corolla is substantially radially symmetric (i.e., the flower shape characteristics of the carnation-like fully double-flowered Torenia plant).

The explanation in the “1. Terminology” section above can be adopted to explain the terms related to breeding and populations in the description about “the progeny population” and “the mutant population” in the paragraphs above. Also, the explanation about “the first-generation hybrid population” and “the subsequent-generation hybrid population derived from the first-generation hybrid population” described in Section 3 below (e.g., (i) to (v) in Section 3) can be adopted to explain the progeny population described above.

### 3. Method for Producing the Double-Flowered Torenia Plant

Certain aspects of the present disclosure relate to methods for producing double-flowered Torenia plants that have the fully double-flowered trait as described above.

### [Fully Double-Flowered Torenia Plant]

A method for producing a Torenia plant having the fully double-flowered trait according to the present disclosure includes the steps of crossing (hybridizing) a Torenia plant having the semi-double-flowered trait and a Torenia plant having the multiple-carpel trait and obtaining the hybrid population.

Specifically, a Torenia plant belonging to the “DF17 strain” (FERM BP-22419), which is the semi-double-flowered strain described in the Examples below, can be used as the Torenia plant having the semi-double-flowered trait that serves as one of the parents of the cross in this method. Also, a Torenia plant whose flower shape is similar to that of the DF17 strain (FERM BP-22419) due to the mutation of the same gene responsible for the mutation in the DF17 strain (FERM BP-22419) can be used as said one of the parents of the cross. Moreover, a semi-double-flowered Torenia plant which belongs to a progeny population or a mutant population of the semi-double-flowered Torenia plant and whose flower has 7 to 10 developed petals can be used as said one of the parents of the cross.

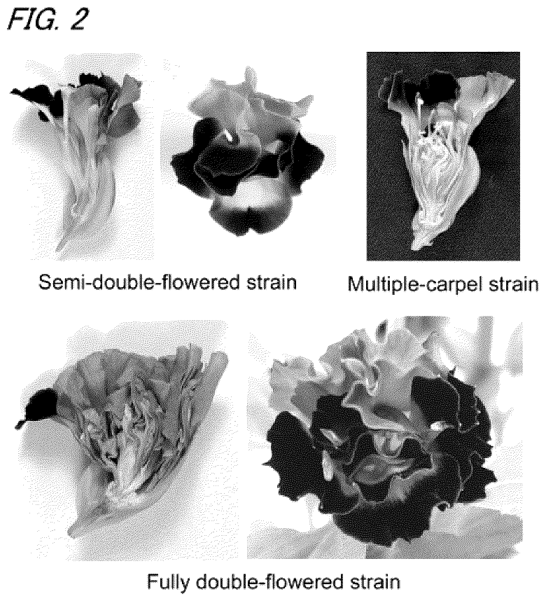

The term “semi-double-flowered” trait as used herein indicates the flower shape trait in which the number of the petals of the flower is slightly increased compared with that of the normal-type Torenia plant and in which the flower has 7 to 10 developed petals (FIG. 2). The increase in the number of the petals of this flower is not sufficient, the petals are arranged sparsely (the gaps between the petals are large), and the flower shape is not enough ornamental as a double-flowered plant. The semi-double-flowered mutation shows a single recessive trait.

Meanwhile, a Torenia plant belonging to the “MC1 strain” (FERM BP-22420), which is the multiple-carpel strain described in the Examples below, can be used as the Torenia plant having the multiple-carpel trait that serves as the other parent of the cross in this method. Also, a Torenia plant whose flower shape is similar to that of the MC1 strain (FERM BP-22420) due to the mutation of the same gene responsible for the mutation in the MC1 strain (FERM BP-22420) can be used as said other parent of the cross. Moreover, a multiple-carpel Torenia plant which belongs to a progeny population or a mutant population of the multiple-carpel Torenia plant and whose flower has an increased number of carpels can be used as said parent of the cross.

The term “multiple-carpel” trait as used herein indicates the flower shape trait in which the number of the carpels constituting the pistil is increased (FIG. 2). The multiple-carpel mutation shows a single recessive trait.

The method for producing the Torenia plant having the fully double-flowered trait according to the present disclosure includes the steps of performing the cross described above (crossing the Torenia plant having the semi-double-flowered trait and the Torenia plant having the multiple-carpel trait) and selecting an individual having the fully double-flowered trait from the offspring population (the second- and subsequent-generation hybrid populations) obtained by the cross.

Here, the fully double-flowered trait of the flower shape in the present disclosure is a phenotype caused by double mutation, i.e., the semi-double-flowered mutation and the multiple-carpel mutation, each of which is governed by a single recessive gene. Therefore, the flower shape with the fully double-flowered trait does not appear in the F1 generation (the first generation of the cross), in which the alleles are heterozygous: it appears in the second and later generations, in which both loci can be homozygous recessive. As a result, individuals with the fully double-flowered trait can be obtained from the second- and subsequent-generation hybrid populations.

The explanation about the “fully double-flowered” trait described in Section 2 above can be adopted to explain the fully double-flowered trait that is used as the basis of selection in the selection step of this production method.

In the production of the fully double-flowered Torenia plant according to the present disclosure, individuals whose flower shape has the fully double-flowered trait can be isolated from the offspring population produced by the crossing step (hybridization) described above.

Here, the offspring population, which is produced in the crossing step described above and is subjected to the selection process, indicates the second- and subsequent-generation hybrid populations. To explain in more detail, examples of the offspring population are the second- and subsequent-generation hybrid populations derived from the first-generation hybrid population, and the first-generation hybrid population is the F1 population that has been obtained.

The term “the first-generation hybrid population” as used herein indicates one or more than one F1 populations obtained by the crossing step (hybridization) described in the present disclosure. In other words, “the first-generation hybrid population” indicates the first-generation population generated as a result of the crossing step (hybridization). In certain embodiments, the “hybridization” includes the production of one or more than one F1 populations by one or more than one crossings between parents described in the paragraphs of the present disclosure. In some such embodiments, the hybridization includes the production of two or more F1 populations by changing the combination of said parents. Meanwhile, the production of the offspring population of the hybrid population includes the production of the offspring population by the cross between two or more different F1 populations (between different populations).

The term “subsequent-generation hybrid population derived from the first-generation hybrid population” as used herein indicates the second- and subsequent-generation hybrid populations. In other words, it indicates the populations which are the second and later generations counted from the crossing step (hybridization) described in the present disclosure.

An example of the “subsequent-generation hybrid population derived from the first-generation hybrid population” includes (i) a population (second-generation hybrid population) produced by the cross within or between one or more than one first-generation hybrid populations (F1 populations), or by the selfing in the first-generation hybrid population (F1 population). The second-generation hybrid population includes the populations produced by the cross between individuals within the same F1 population, the cross between individuals of two different F1 populations, and the selfing of an individual that belongs to the F1 population.

Another example of the “subsequent-generation hybrid population derived from the first-generation hybrid population” includes (ii) a population (third-generation hybrid population) produced by the cross within the second-generation hybrid population, by the cross between the second-generation hybrid populations, or by the selfing in the second-generation hybrid population. The third-generation hybrid population includes the populations produced by the cross between individuals within the same second-generation hybrid population, the cross between individuals of two different second-generation hybrid populations, and the selfing of an individual that belongs to the second-generation hybrid population.

Another example of the “subsequent-generation hybrid population derived from the first-generation hybrid population” includes (iii) a population (fourth generation hybrid population) produced by the cross within the third-generation hybrid population, by the cross between the third-generation hybrid populations, or by the selfing in the third-generation hybrid population. The fourth generation hybrid population includes the populations produced by the cross between individuals within the same third-generation hybrid population, the cross between individuals of two different third-generation hybrid populations, and the selfing of an individual that belongs to the third-generation hybrid population.

The “subsequent-generation hybrid population derived from the first-generation hybrid population” also includes a population produced by the cross within the population of the preceding generation, by the cross between the populations of the preceding generation, or by the selfing in the population of the preceding generation, when the breeding process is repeated to produce further generations. That is, the “subsequent-generation hybrid population derived from the first-generation hybrid population” includes the offspring populations produced by performing the crossing step (hybridization) and repeating the breeding step, which is conducted by the cross within the population of the preceding generation, the cross between the populations of the preceding generation, or the selfing within the population of the preceding generation, for generations. Its examples include (iv) populations that are the fifth or subsequent generations (fifth- or subsequent-generation hybrid populations) counted from the crossing step (hybridization) described above.

Moreover, the “subsequent-generation hybrid population derived from the first-generation hybrid population” includes a population produced by the cross between the populations of different generations counted from the crossing step (hybridization) described above. Its examples include (v) a population produced by the cross between the first-generation hybrid population and the second-generation hybrid population. They also include a population produced by the cross between the first-generation hybrid population and the third-generation hybrid population. They further include a population produced by the cross between the second-generation hybrid population and the third-generation hybrid population.

Also, the “subsequent-generation hybrid population derived from the first-generation hybrid population” includes a population further produced by the cross between the populations that have been obtained by the cross between different generations described above. The “subsequent-generation hybrid population derived from the first-generation hybrid population” also includes a population produced by the cross between the population of any of the generations described above and the population that has been obtained by the cross between the populations of different generations described above.

The term “a cross within a population” as used herein refers to a cross between individuals that belong to the same population. The term “a cross between populations” refers to a cross between individuals that belong to two different populations. The cross includes random mating between individuals. The term “a selfing” refers to reproduction by self-pollination of an individual.

The population that is subjected to selection can be the population of the fourth or earlier generation of the cross, or preferably, the population of the third or earlier generation of the cross, so that any unfavorable traits may not be expressed due to inbreeding.

The explanation in the “1. Terminology” section above can be adopted to explain the terms related to breeding and the population of each generation.

The selection step in this production method includes the step of selecting an individual whose flower shape has the fully double-flowered trait, that is, whose flowers have a sufficient number of developed petals (examples of the number of petals is described above). The details about the “fully double-flowered” trait described in Section 2 above can be adopted to explain the fully double-flowered trait that is used as the basis of selection in this selection step.

The selection step of this production method enables to obtain Torenia plants whose flower shape has the fully double-flowered trait.

The present disclosure further enables to produce a Torenia plant that has a desired trait as well as the fully double-flowered trait through the usage of the fully double-flowered Torenia plant obtained by the steps described above as a parent of the cross.

The method for producing the double-flowered Torenia plant in this embodiment includes the steps of crossing (hybridizing) the Torenia plant whose flower shape has the fully double-flowered trait described above with a freely selected Torenia plant and obtaining the hybrid population.

The method for producing the double-flowered Torenia plant having a desired trait in this embodiment includes the step of, after the crossing (between the Torenia plant whose flower shape has the fully double-flowered trait and the freely selected Torenia plant), selecting an individual that has a desired trait as well as the fully double-flowered trait from the offspring populations (second- and subsequent-generation hybrid populations) derived from the crossing.

The preferable ranges of the offspring populations (the second- and subsequent-generation hybrid populations) to be subjected to the selection step in the production method in this embodiment can be determined based on the descriptions in the paragraphs above. Also, the explanation in the “1. Terminology” section above and the explanation about “the first-generation hybrid population” and “the subsequent generation hybrid population derived from the first-generation hybrid population” described in Section 3 (e.g., (i) to (v) described above) can be adopted to explain the terms about breeding and the population of each generation. Moreover, the explanation about the “fully double-flowered” trait described in Section 2 above can be adopted to explain the fully double-flowered trait that is used as the basis of selection in the selection step of this production method.

In this embodiment, the selection step makes it possible to obtain a Torenia plant that has a desired trait inherited from the one parent of the cross (the freely selected Torenia plant) in addition to the fully double-flowered trait. Moreover, the selection step in this embodiment makes it possible to obtain a Torenia plant having a desired trait that is newly expressed as a result of the combination of the parent plants (the fully double-flowered Torenia plant and the freely selected Torenia plant).

In the present disclosure, a Torenia plant whose flower shape has the fully double-flowered trait can also be obtained by further performing the cross (hybridization) between two parents both of which are the fully double-flowered Torenia plants obtained in the steps described above. In this embodiment, it can be also possible to select and produce a fully double-flowered Torenia plant having a desired trait through the cross between two types of fully double-flowered Torenia plants having different characteristics and traits.

In this embodiment of producing the double-flowered Torenia plant, individuals with the fully double-flowered trait can be obtained from the first- and subsequent-generation hybrid populations because both loci described above can be homozygous recessive in the first and subsequent generations.

That is, in the production of the double-flowered Torenia plant in this embodiment, it becomes possible to select an individual having the fully double-flowered trait from the hybrid population (the first-generation hybrid population obtained by the cross: F1 population) or from its offspring population (second- or subsequent-generation hybrid population) that are obtained as a result of the cross (the cross between the fully double-flowered Torenia plants described above).

The preferable ranges and conditions in the selection step can be determined based on the descriptions in the paragraphs above. Also, the explanation in the “1. Terminology” section above and the explanation about “the first-generation hybrid population” and “the subsequent-generation hybrid population derived from the first generation hybrid population” described in Section 3 (e.g., (i) to (v) described above) can be adopted to explain the terms about breeding and the population of each generation. Moreover, the explanation about the “fully double-flowered” trait described in Section 2 above can be adopted to explain the fully double-flowered trait that is used as the basis of selection in the selection step of this production method.

### [Carnation-Like Fully Double-Flowered Torenia Plant]

An embodiment of the fully double-flowered Torenia plant according to the present disclosure relates to the carnation-like fully double-flowered Torenia plant whose flower shape has even higher ornamental value. In this respect, the present disclosure enables to produce a Torenia plant having the carnation-like fully double-flowered trait through the usage of the fully double-flowered Torenia plant as a parent of the cross.

A method for producing the carnation-like fully double-flowered Torenia plant according to the present disclosure includes the steps of crossing (hybridizing) the Torenia plant whose flower shape has the fully double-flowered trait and the Torenia plant whose flower shape has the begonia-like trait and of obtaining the hybrid population.

The fully double-flowered Torenia plant produced in the production method described above can be used as the “Torenia plant whose flower shape has the fully double-flowered trait”, which serves as one of the parents of the cross. It is also possible to use the Torenia plant that belongs to the 11118strain (FERM BP-22437), which is the fully double-flowered strain described in the Examples section below. Moreover, the Torenia plant which belongs to the subsequent or mutant population of the aforementioned Torenia plant (i.e., the fully double-flowered Torenia plant produced in the production method described above or the Torenia plant that belongs to the 11118 strain (FERM BP-22437)) and whose flower shape has the fully double-flowered trait can be used as said one of the parents of the cross.

Meanwhile, the Torenia plant that belongs to the RSF1 strain (FERM BP-22422), which is the uniformly pigmented begonia-like Torenia strain described in the Examples section below, can be used as the “Torenia plant whose flower shape has the begonia-like trait”, which serves as the other parent of the cross. Also, the Torenia plant whose flower shape is similar to that of the RSF1 strain (FERM BP-22422) due to the mutation of the same gene as in the RSF1 strain (FERM BP-22422) and yet whose flower is not uniformly pigmented (Non-Patent Document 3: Niki et al., 2016) can be used as said other parent of the cross. In a preferred embodiment, the Torenia plant which belongs to the FERM BP-22422 strain or to the population derived from this strain can be used, because they have petals pigmented uniformly.

Also, the Torenia plant which belongs to the subsequent or mutant population of the begonia-like Torenia plant and whose flower shape is both bilaterally and dorsoventrally symmetric can be used as said other parent of the cross.

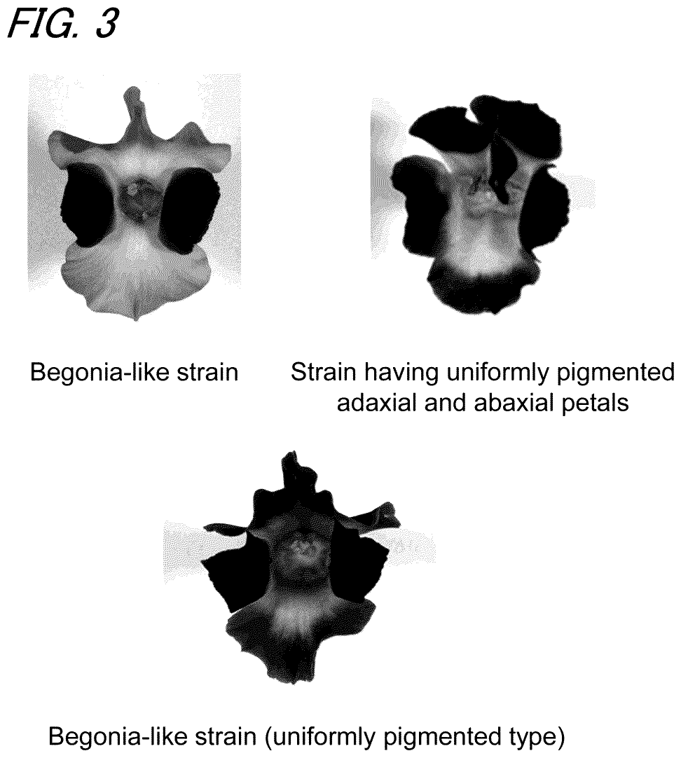

As used herein, the “begonia-like” trait indicates the flower shape trait in which, in addition to the bilateral symmetry, which is a trait of the normal-type Torenia flower, the adaxial (upper) and abaxial (lower) petals are also symmetric to each other because the abaxial (lower) petal is converted into the adaxial (upper) petal (FIG. 3). Though the number of the axes of symmetry in this flower shape is increased compared with the flower shape of the normal-type Torenia plant, the corolla is not substantially radially symmetric. The begonia-like mutation shows a single recessive trait.

The method for producing the Torenia plant having the carnation-like fully double-flowered trait according to the present disclosure includes the steps of performing the cross described above (crossing the Torenia plant having the fully double-flowered trait and the Torenia plant having the begonia-like trait) and selecting an individual having the carnation-like fully double-flowered trait from the offspring population (the second- or subsequent-generation hybrid population) obtained by the cross.

Here, the “carnation-like fully double-flowered” trait of the flower shape in the present disclosure is a phenotype derived from the triple mutation, i.e., the semi-double-flowered mutation, the multiple-carpel mutation, and the begonia-like mutation, each of which is caused by a single recessive allele. Thus, the flower shape with the carnation-like fully double-flowered trait does not appear in the F1 generation (the first generation of the cross), in which the alleles are heterozygous. This flower shape appears in the second and subsequent generations, in which the genotypes at all of the three loci can be homozygous recessive. Therefore, individuals with the carnation-like fully double-flowered trait can be obtained from the second- and subsequent-generation hybrid populations.

In the production of the fully double-flowered Torenia plant according to the present disclosure, individuals whose flower shape has the carnation-like fully double-flowered trait can be isolated from the offspring population produced by the crossing step (hybridization) described above.

Here, the offspring population, which is produced in the crossing step described above (cross between the Torenia plant whose flower shape has the fully double-flowered trait and the Torenia plant whose flower shape has the begonia-like trait) and is subjected to the selection process, indicates the second- and subsequent-generation hybrid populations obtained through the crossing. To explain in more detail, the examples of the offspring population are the second- and subsequent-generation hybrid populations derived from the first-generation hybrid population, the first-generation hybrid population being the F1 population that has been obtained.

The preferable ranges of the offspring populations (the second- and subsequent-generation hybrid populations) to be subjected to the selection step in this production method can be determined based on the description in the paragraphs above. Also, the explanation in the “1. Terminology” section above and the explanation about “the first-generation hybrid population” and “the subsequent-generation hybrid population derived from the first-generation hybrid population” described in Section 3 (e.g., (i) to (v) described above) can be adopted to explain the terms about breeding and the population of each generation.

Moreover, the explanation about the “carnation-like fully double-flowered” trait described in Section 2 above can be adopted to explain the carnation-like fully double-flowered trait that is used as the basis of selection in the selection step of this production method.

The population that is subjected to selection can be the population of the fourth or earlier generation of the cross, or preferably, the population of the third or earlier generation of the cross, so that any unfavorable traits may not be expressed due to inbreeding.

The selection step in the production method includes the step of selecting an individual that has the carnation-like fully double-flowered trait, i.e., an individual whose flower has a sufficient number of developed petals (examples of the number of petals are described above) that are arranged radially around the floral axis to form a double flower with a substantially radially symmetric corolla. The details about the “carnation-like fully double-flowered” trait described in Section 2 above can be adopted to explain the carnation-like fully double-flowered trait that is used as the basis of selection in this selection step.

The selection step in this production method enables to obtain Torenia plants whose flower shape has the carnation-like fully double-flowered trait.

The present disclosure further enables to produce a Torenia plant that has a desired trait as well as the carnation-like fully double-flowered trait through the usage of the carnation-like fully double-flowered Torenia plant obtained by the method described above as a parent of the cross.

The method for producing the double-flowered Torenia plant in this embodiment includes the steps of crossing (hybridizing) the Torenia plant whose flower shape has the carnation-like fully double-flowered trait described above with a freely selected Torenia plant and obtaining the hybrid population.

The method for producing the double-flowered Torenia plant having the desired trait in this embodiment includes the step of, after the crossing (between the Torenia plant whose flower shape has the carnation-like fully double-flowered trait and the freely selected Torenia plant), selecting an individual that has the desired trait as well as the carnation-like fully double-flowered trait from the offspring populations (the second- and subsequent-generation hybrid populations) derived from the crossing.

The preferable ranges of the offspring populations (the second- and subsequent-generation hybrid populations) to be subjected to the selection step in this production method can be determined based on the description in the paragraphs above. Also, the explanation in the “1. Terminology” section above and the explanation about “the first-generation hybrid population” and “the subsequent-generation hybrid population derived from the first-generation hybrid population” described in Section 3 (e.g., (i) to (v) described above) can be adopted to explain the terms about breeding and the population of each generation. Moreover, the explanation about the “carnation-like fully double-flowered” trait described in Section 2 above can be adopted to explain the carnation-like fully double-flowered trait that is used as the basis of selection in the selection step of this production method.

In this embodiment, the selection step makes it possible to obtain a Torenia plant that has a desired trait inherited from the one parent of the cross (the freely selected Torenia plant) in addition to the carnation-like fully double-flowered trait. Moreover, the selection step in this embodiment makes it possible to obtain a Torenia plant having a desired trait that is newly expressed as a result of the combination of the parent plants (the carnation-like fully double-flowered Torenia plant and the freely selected Torenia plant).

In the present disclosure, usage of the carnation-like fully double-flowered Torenia plants obtained in the method described above as both parents of the cross enables to further produce a Torenia plant whose flower shape has the carnation-like fully double-flowered trait. In this embodiment, the cross (hybridization) between two types of carnation-like fully double-flowered Torenia plants having different characteristics and traits also makes it possible to obtain a carnation-like fully double-flowered Torenia plant having a desired trait.

In the production of the double-flowered Torenia plant in this embodiment, individuals with the carnation-like fully double-flowered trait can be obtained from the first- and subsequent-generation hybrid populations of the cross because individuals having homozygous recessive genotypes at all of the three loci can appear in the first and subsequent generations.

That is, in the production of the double-flowered Torenia plant in this embodiment, it becomes possible to select an individual having the carnation-like fully double-flowered trait from the hybrid population (the first-generation hybrid population obtained by the cross: F1 population) or from its offspring population (second- or subsequent-generation hybrid population) that are obtained as a result of the cross (the cross between the carnation-like fully double-flowered Torenia plants as described above).

The preferable ranges in the selection step can be determined based on the description in the paragraphs above. Also, the explanation in the “1. Terminology” section above and the explanation about “the first generation hybrid population” and “the subsequent-generation hybrid population derived from the first-generation hybrid population” described in Section 3 (e.g., (i) to (v) described above) can be adopted to explain the terms about breeding and the population of each generation. Moreover, the explanation about the “carnation-like fully double-flowered” trait described in Section 2 above can be adopted to explain the carnation-like fully double-flowered trait that is used as the basis of selection in the selection step of this production method.

### 4. Other Aspects of the Disclosure

### [Plant Body]

An aspect of the present disclosure relates to the plant body of the Torenia plant having the characteristics described above.

The plant body of the double-flowered Torenia plant according to the present disclosure can include all the tissues and organs constituting the plant body of the Torenia plant describe above. It can also include any part of the plant body constituting the entire or a part of the plant body. It can also include tissues, organs, and the like, of the plant body. It can also include plant bodies in any growth stages.

Examples of the plant body are a flower (floral part), an organ constituting the flower (e.g. a sepal, a petal, a stamen, and a pistil), a bud, a flower bud, a leaf, a stem, a sprout, a fruit, a seed, and a root. The plant body can be in any growth stages, and its examples are a young seedling, a seedling, a plant body in a vegetative growth stage, a plant body in a flower budding stage, a plant body having blooming flowers, and a plant body after the flowers have withered. A plant body reproduced vegetatively is also included.

Moreover, a plant body under cultivation, such as a cultured cell, a callus cell, and a cultured tissue, is also included.

The plant body of the Torenia plant in one embodiment of the present disclosure is a living plant body. Its examples are a plant body planted in the ground, a plant body planted in a pot, a plant body grown from a cutting, and a plant body planted in an artificial medium.

The plant body in another embodiment is a part of a plant body that has been harvested or cut. Its examples are a cut flower, a bouquet, and the like. The plant body in still another embodiment includes a product obtained by processing a part of a plant body. Its examples are a dried flower, a pressed flower, a preserved flower, and a plant body embedded in resin.

### [Method for Producing a Torenia Strain]

The present disclosure enables to obtain a Torenia plant having characteristics described above, and moreover, to produce a Torenia strain through the usage of this Torenia plant.

That is, one aspect of the present disclosure relates to a double-flowered Torenia strain whose population includes or is composed of the double-flowered Torenia plants described above.

Another aspect of the present disclosure relates to a method for producing a Torenia strain which has the Torenia plants described above as the individuals constituting the population. In this method, steps such as selfing, crossing between individuals of the same strain, crossing, and/or selection can be conducted or repeated, a population of the double-flowered Torenia plants having characteristics and traits described above can be obtained, and a desired Torenia strain can be produced. The method can also include a vegetative reproduction step. The description in the paragraphs above and the Examples section shows the concrete details of these steps.

The term “strain” as used herein refers to a subgroup of a species and is used to indicate a population having morphological and/or physiological characteristics (phenotype) that can be distinguished from those of other cultivars or strains. Meanwhile, among strains, a population in which the characteristics of individuals in a generation have uniformity, being sufficiently similar to each other, and in which the characteristics are maintained stably through generations is called a “cultivar”. Strains also include cultivars.

### [Various Methods]

Certain further aspects of the present disclosure include various methods related to the Torenia plants having characteristics described above.

An aspect of the present disclosure relates to a method for producing a double-flowered Torenia plant whose flower shape has the characteristics described above. Another aspect of the present disclosure relates to a method for cultivating a double-flowered Torenia plant whose flower shape has the characteristics described above. Still another aspect of the present disclosure relates to a method for producing a plant body of a double-flowered Torenia plant whose flower shape has the characteristics described above. Yet another aspect of the present disclosure relates to a method for producing a strain or a variety of a double-flowered Torenia plant whose flower shape has the characteristics described above.

These methods include the step of using a seed, a seedling, a plant tissue or the like of the Torenia plant provided in the present disclosure. In these methods, any ordinary procedures can be employed and conducted under any ordinary conditions if the Torenia plant according to the present disclosure is used.

### EXAMPLES

Hereinafter, exemplary embodiments of the present invention will be described, but the invention is not limited thereto.

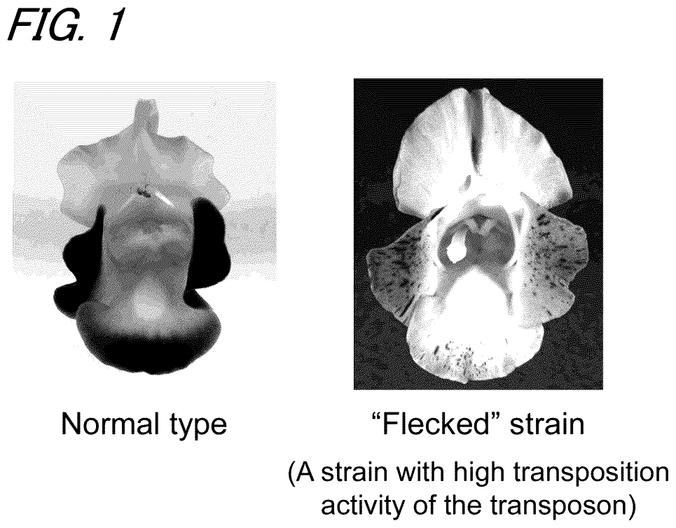

A Torenia mutable line “flecked” (FIG. 1) having high transposition activity of the transposon was discovered by the present inventor from mutants obtained by chemical mutagen treatment. The present inventor developed a method to obtain various Torenia mutants through selection of selfed progeny of the “flecked”. The “flecked” strain and the method for producing mutants through the usage of the transposition activity of the transposon are reported by the present inventor in Non-Patent Document 2. However, there has been no report on highly ornamental, double-flowered Torenia plants obtained through screening and selection of the selfed progeny of the “flecked”.

In the examples below, each “F2 population” produced indicates a population (second-generation hybrid population) produced by selfing of the individuals that belong to an F1 population obtained in a crossing experiment.

### Example 1

### Production of Torenia Plants With the Fully Double-Flowered Trait

The present inventor produced highly ornamental, double-flowered Torenia plants having a sufficient number of developed petals.

### (1) Production of the Semi-Double-Flowered Strain

The inventor conducted screening and selection of the selfed progeny of the “flecked” with focus on the flower shape characteristics and found a semi-double-flowered mutant having a slightly increased number of petals. The inventor selected a strain having stable flower characteristics from the selfed progeny of this mutant and produced the DF17 strain (FERM BP-22419) as the semi-double-flowered strain. Though the flower of this semi-double-flowered Torenia strain had more petals than the normal-type Torenia flower, the number of petals was as low as 7 to 10 and it was hard to conclude that this Torenia strain was a highly ornamental double-flowered strain (FIG. 2).

The inventor performed a test cross between the semi-double-flowered strain (FERM BP-22419) and the normal-type, single-flowered Torenia plant, and all the 52 individuals in the F1 generation had the normal-type flower shape. In the F2 generation, 133 individuals (73%) were the normal type, and 48 individuals (27%) were the semi-double-flowered mutant type. This segregation ratio is statistically consistent with the theoretical segregation ratio of 25%, which is calculated under the hypothesis that the semi-double-flowered mutation is controlled by a single-recessive gene. This result shows that the semi-double-flowered trait is inherited by a single recessive gene.

### (2) Production of the Multiple-Carpel Strain

In addition to the semi-double-flowered strain described above, the inventor found a multiple-carpel mutant through screening and selection of the selfed progeny of the “flecked”. The multiple-carpel mutant has an increased number of carpels constituting the pistil, while it does not have an increased number of petals (FIG. 2). The inventor selected a strain having stable flower characteristics from the selfed progeny of this mutant and produced the MC1 strain (FERM BP-22420) as the multiple-carpel strain.

The inventor performed a test cross between the multiple-carpel strain (FERM BP-22420) and the normal-type, single-flowered Torenia plant, and all the 48 individuals in the F1 generation had the normal-type flower shape. In the F2 generation, 118 individuals (74%) were the normal type, and 42 individuals (26%) were the multiple-carpel mutant type. This segregation ratio is statistically consistent with the theoretical segregation ratio of 25%, which is calculated under the hypothesis that the multiple-carpel trait is controlled by a single-recessive gene. This result shows that the multiple-carpel trait is inherited by a single recessive gene.

### (3) Production of the Fully Double-Flowered Strain

Through the screening and selection process conducted on the selfed population of the “flecked” described above, it was impossible to obtain any double-flowered Torenia mutants having more petals than the semi-double-flowered strain described in (1) above.

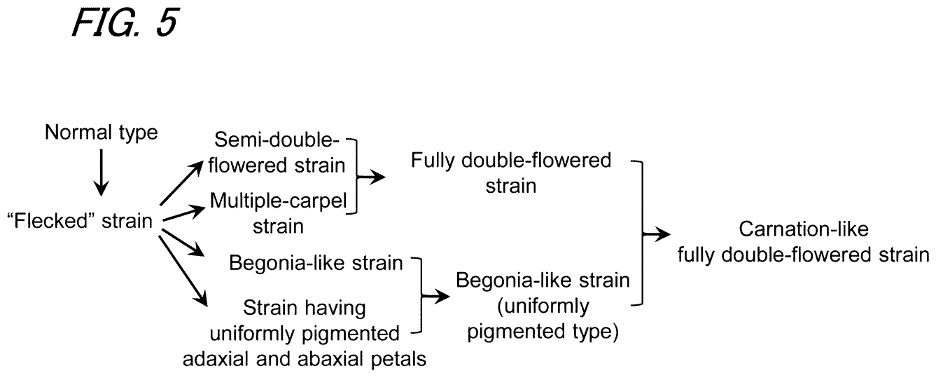

Accordingly, the semi-double-flowered strain (FERM BP-22419) and the multiple-carpel strain (FERM BP-22420) were crossed, and flower shapes expressed in progeny populations were observed (FIGS. 2 and 5). In this experiment, the semi-double-flowered strain was used as the pollen parent, and the multiple-carpel strain was used as the seed parent.

The results of the cross were as follows. All the 36 individuals in the F1 generation had the normal-type flower shape. Meanwhile, among the 153 individuals in the F2 generation, 9 individuals (5.9%) were fully double-flowered (FIG. 2). This value is statistically consistent with the theoretical value of 6.25% calculated as the frequency of the double mutant having both of the semi-double-flowered mutation and the multiple-carpel mutation, each governed by a single recessive gene.

The inventor obtained a population with stable flower characteristics from the fully double-flowered Torenia plants generated and produced the 11118 strain (FERM BP-22437) as the fully double-flowered strain.

These results show that, from the F2 population obtained by crossing the semi-double-flowered strain and the multiple-carpel strain, fully double-flowered Torenia plants having a sufficient number of developed petals can be obtained at a certain frequency as the double mutant that has both of the semi-double-flowered mutation and the multiple-carpel mutation. The fully double-flowered Torenia plants obtained in this production step had a remarkably increased number of developed petals: the number of petals was within the range of 19 to 50 (in many cases, it was within the range of 19 to 33). These Torenia plants were highly ornamental, bearing double flowers with frilled spatulate petals (FIG. 2).

The size of the developed petals when fully expanded was as follows: the petal length (the length from the basal part including the tubular part to the upper end of the petal) was 20 to 45 mm, the lobe length (the length without the tubular part) was 5 to 15 mm, and the petal width (the width at the widest part of the petal) was 5 to 20 mm.

It should be noted that the multiple-carpel mutation of the multiple-carpel strain, which is one of the parents of the cross, is a pistil mutation. The crossing experiment in this example has proved, for the first time ever, that the number of developed petals can be remarkably increased in the double mutant obtained by crossing the multiple-carpel strain and the semi-double-flowered strain.

Fully double-flowered individuals whose flowers had 19 or more developed petals were obtained from the F2 population in this example. Yet, the number of petals may vary to some extent even in a population having the same genetic background. Also, in case of Torenia, if the flower has 15 or more developed petals, the petals can be arranged densely and radially around the floral axis, and the Torenia plant can be regarded as being fully double-flowered.

Therefore, even the individuals which have the same genetic background as the fully double-flowered Torenia plant described above and yet whose flowers have less than 19 petals due to some reasons can be regarded as the fully double-flowered Torenia plants according to the present disclosure, if their flowers have 15 or more developed petals.

With regard to the shape of the petal in each region of the flower of the fully double-flowered strain produced in this example, the spatulate shape of the adaxial petals and the spatulate shape of the abaxial and lateral petals are different from each other, just like those of the normal-type Torenia flower. Moreover, the adaxial petals, the abaxial petals, and the lateral petals are located in the same respective regions as those of the normal-type Torenia flower. Therefore, even though the flower is fully double-flowered, the shape of the corolla is not substantially radially symmetric.

The adaxial petal of this fully double-flowered strain is entirely pale violet, just like the adaxial petal of the normal-type Torenia flower. The color of the abaxial and lateral petals is pale violet from the basal part to the middle part and is dark violet from the middle part to the upper part of the petal. The abaxial petal has a yellow nectar guide in its central region. In these regards, the flower of the fully double-flowered strain produced in this example is not pigmented in a substantially radially symmetric manner, either.

### Example 2

### Production of Torenia Plants With the Carnation-Like Fully Double-Flowered Trait

The inventor produced double-flowered Torenia plants bearing flowers with higher ornamental value, using the fully double-flowered strain produced in Example 1 above as the parental line.

### (1) Production of the Begonia-Like Strain

In addition to the Torenia strains described in the example above, the inventor found the begonia-like mutant through screening and selection of the selfed progeny of the “flecked”. The flower of the begonia-like mutant had bilateral symmetry, which is a characteristic of the normal-type Torenia flower, and moreover, it had dorsoventral symmetry (symmetric about a horizontal line), with the abaxial (lower) petals converted into the adaxial (upper) petals. When compared with the shape of the normal-type Torenia flower, which is only bilaterally symmetric, the flower shape of the begonia-like mutant is symmetric both bilaterally and dorsoventrally. A population having stable flower characteristics was obtained by removing the transposition activity of the transposon, and the begonia-like strain was produced (Non-Patent Document 3: Niki et al., 2016) (FIG. 3).

The begonia-like mutation is reported to be governed by a single recessive gene (Non-Patent Document 3: Niki et al., 2016).

### (2) Production of the Strain Having Uniformly Pigmented Adaxial and Abaxial Petals

In addition to the Torenia strains described in the example above, the inventor found a mutant having uniformly pigmented adaxial and abaxial petals through screening and selection of the selfed progeny of the “flecked”. This mutant had adaxial and abaxial petals that were pigmented in a similar way to the lateral petals (whose basal part is pigmented in pale violet and whose upper part is pigmented in dark violet), and as a result, all the petals of this mutant were pigmented uniformly. A population having stable flower characteristics was obtained by removing the transposition activity of the transposon, and the strain having uniformly pigmented adaxial and abaxial petals was produced (Non-Patent Document 4: Nishijima et al., 2015) (FIG. 3).

It has been proved that the mutation of the TfRAD1 gene is responsible for the mutant trait of the strain having uniformly pigmented adaxial and abaxial petals (Nishijima et al., 2015), which is inherited as a recessive trait controlled by a single gene. This strain having uniformly pigmented adaxial and abaxial petals is commercially available at the time of the filing of this application.

### (3) Production of the Begonia-Like Strain (Uniformly Pigmented Type)

The inventor conducted a cross between the begonia-like strain, which was produced in the example above, and the strain having uniformly pigmented adaxial and abaxial petals, which was obtained as a commercially available cultivar, and investigated the flower shapes of the hybrids in the progeny populations (FIGS. 3 and 5).

The results of the cross were as follows. All the individuals in the F1 generation had the normal-type flower shape. In the F2 generation, 4.8% of the individuals produced bore flowers whose flower shape was bilaterally and dorsoventrally symmetric (like a begonia flower) and all of whose petals were pigmented in a similar manner (FIG. 3). This result is statistically consistent with the theoretical value of 6.25% calculated as the frequency of a double mutation in the F2 generation occurred by the combination of mutations (the begonia-like mutation and the mutation expressing uniformly pigmented adaxial and abaxial petals) each governed by a single recessive gene.

From the uniformly pigmented begonia-like Torenia plants obtained, the inventor obtained a population having stable flower characteristics and produced the RSF1 strain (FERM BP-22422) as the uniformly pigmented begonia-like strain.

These results show that, from the F2 population obtained as a result of the cross between the begonia-like strain and the strain having uniformly pigmented adaxial and abaxial petals, the begonia-like Torenia plant having uniformly pigmented petals, which is the double mutant produced by the combination of these strains, can be obtained at a certain frequency. The uniformly pigmented begonia-like Torenia plant obtained in this production step has highly ornamental flower characteristics, bearing flowers whose flower shape is bilaterally and dorsoventrally symmetric and all of whose petals are pigmented in a similar manner (the pigmentation patterns of the petals do not vary, i.e., all the petals, including the adaxial and abaxial petals and the lateral petals, are pigmented in pale violet at the basal part and pigmented in dark violet at the upper part) (FIG. 3). Meanwhile, the flower of this uniformly pigmented begonia-like Torenia plant has bilateral symmetry, which is a characteristic of the normal-type Torenia flower, and in addition, it has dorsoventral symmetry as a result of the effects of the begonia-like mutation. Yet, it does not have a substantially radially symmetric corolla.

### (4) Production of the Carnation-Like Fully Double-Flowered Strain

The fully double-flowered strain produced in Example 1 above bears highly ornamental double flowers with a sufficiently large number of developed petals. The inventor further produced Torenia plants bearing flowers with even higher ornamental value, using the fully double-flowered strain produced in Example 1 above and the uniformly pigmented begonia-like strain (which has the begonia-like flower shape characteristics and all of whose petals are pigmented uniformly) produced in (3) above as the parental lines.

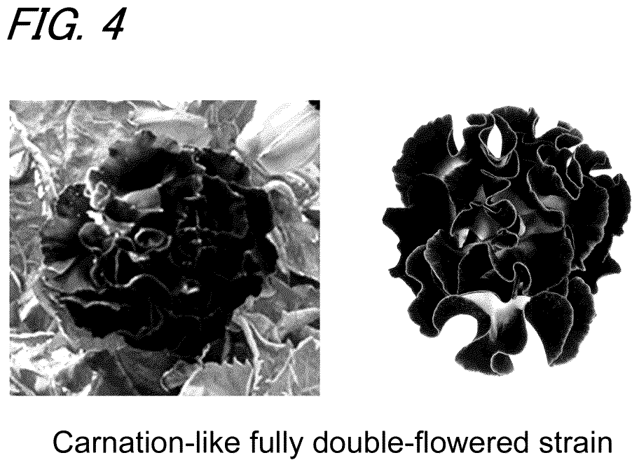

The inventor conducted a cross between the fully double-flowered strain (FERM BP-22437) and the begonia-like strain (uniformly pigmented type) (FERM BP-22422) that had been produced in the examples above and investigated the flower shapes of the hybrids in the progeny populations (FIGS. 3, 4 and 5). In this crossing experiment, the fully double-flowered strain was used as the seed parent, and the uniformly pigmented begonia-like strain was used as the pollen parent.

The results of the cross were as follows. All the individuals in the F1 generation had the normal-type flower shape. In the F2 generation, 0.8% of the individuals produced had the fully double-flowered trait, having flowers with a sufficiently large number of developed petals, and moreover, had petals arranged radially around the floral axis to form a double flower with a substantially radially symmetric corolla (FIG. 5). The flower shape was like that of a carnation. Moreover, all the petals were pigmented in a similar manner.

The frequency of this uniformly pigmented, carnation-like, fully double-flowered trait is statistically consistent with the theoretical value of 0.4% calculated as the frequency of the quadruple mutation in the F2 generation occurred by the combination of the “semi-double-flowered mutation”, the “multiple-carpel mutation”, the “begonia-like mutation”, and the “mutation expressing uniformly pigmented adaxial and abaxial petals”, each of which is governed by a single recessive gene. Meanwhile, the “carnation-like fully double-flowered” mutation (i.e., the flower shape mutation, with the pigmentation pattern not taken into consideration) is considered to be a phenotype of the triple mutation occurred by the combination of the “semi-double-flowered mutation”, the “multiple-carpel mutation”, and the “begonia-like mutation”.

A population with stable flower characteristics was obtained from the carnation-like fully double-flowered Torenia plants generated, and the 10868 strain (FERM BP-22418) was produced as the carnation-like fully double-flowered strain. In this strain, all the petals of the flower are pigmented in a similar manner (the pigmentation patterns of the petals do not vary, i.e., all the petals are pigmented in pale violet at the basal part and pigmented in dark violet at the upper part).

These results show that the Torenia plants which bore flowers having the fully double-flowered trait, and moreover, in which the petals of a flower are arranged radially around the floral axis to form a double flower with a substantially radially symmetric corolla (i.e., the Torenia plants having the carnation-like flower shape) can be obtained at a certain frequency from the F2 population produced by the cross between the fully double-flowered strain and one strain of the begonia-like strains.

The carnation-like fully double-flowered Torenia plants obtained in this production step had highly ornamental flower shape characteristics: their flower had a remarkably increased number of developed petals, which was within the range of 19 to 50 (19 to 33 petals in most frequent cases), the petals constituting the flower were frilled and arranged radially, and they bore substantially radially symmetric double flowers (FIG. 5). While their flower had two different types of frilled spatulate petals (one type corresponding to the lateral petals of the normal-type flower and the other type corresponding to the adaxial petal of the normal-type flower), these petals were randomly and radially arranged, and consequently, the corolla was substantially radially symmetric. The size of the developed petals when fully expanded was as follows: the petal length (the length from the basal part including the tubular part to the upper end of the petal) was 20 to 45 mm, the lobe length (the length without the tubular part) was 5 to 15 mm, and the petal width (the width at the widest part of the petal) was 5 to 20 mm. When viewed facing the front face of the flower, the flower had a substantially radially symmetric corolla, which could not be observed in the normal Torenia plant.

The carnation-like fully double-flowered individuals whose flower had 19 or more developed petals were obtained from the F2 population in this example. Yet, the number of petals may vary even in a population having the same genetic background. Also, in case of Torenia, if the flower has 15 or more developed petals, the petals can be arranged densely and radially around the floral axis, and the Torenia plant can be regarded to be fully double-flowered.

Therefore, even the individuals which have the same genetic background as the carnation-like fully double-flowered Torenia plant described above and yet whose flowers have less than 19 petals due to some reasons can be regarded as the carnation-like fully double-flowered Torenia according to the present disclosure, if their flowers have 15 or more developed petals.

The fully double-flowered strain, which is one of the parents of the cross, bears double flowers, but its adaxial, abaxial and lateral petals only exist at their respective positions in the flower, just like those of the normal-type Torenia flower. Consequently, the corolla of the fully double-flowered strain is not substantially radially symmetric. Also, while the adaxial and abaxial petals of the uniformly pigmented begonia-like strain have the same shape, the shape of the petals located in the dorsoventral direction (the adaxial and abaxial petals) is different from the shape of the petals located in the bilateral direction (the lateral petals). As a result, the corolla of the uniformly pigmented begonia-like strain is not substantially radially symmetric. In these regards, this crossing experiment has proved for the first time ever that the carnation-like flower having the substantially radially symmetric corolla (a flower shape which is approximately symmetric about any radial axis passing through the floral axis when viewed facing the front face of the flower) can be obtained by the production of the multiple mutants through the cross between the fully double-flowered strain and the begonia-like strain.

### [Reference to Deposited Biological Materials]

The double-flowered Torenia strains produced in the production steps described above and their parental strains used in these production steps were deposited with a depositary institution. The Torenia strains described below were deposited with the International Patent Organism Depositary, National Institute of Technology and Evaluation and were given respective accession numbers.

A request to convert the original deposit to a deposit under the Budapest Treaty was received by the International Depositary Authority on 2 Dec. 2022, and the DF17 strain (semi-double-flowered strain) was given the international accession number FERM BP-22419, the MC1 strain (multiple-carpel strain) was given the international accession number FERM BP-22420, the 11118 strain (fully double-flowered strain) was given the international accession number FERM BP-22437, the RSF1 strain (uniformly pigmented begonia-like strain) was given the international accession number FERM BP-22422, and the 10868 strain (carnation-like fully double-flowered strain) was given the international accession number FERM BP-22418. The Receipt in the Case of an Original Deposit was issued for each of these biological materials on 6 Feb. 2023.

### (1) Name and Address of the Depositary Institution (International Depositary Authority)

Name: International Patent Organism Depositary, National Institute of Technology and Evaluation
Address: #120, 2-5-8 Kazusakamatari, Kisarazu-shi, Chiba 292-0818, Japan

### (2) Accession Numbers and Related Dates

FERM BP-22418: Torenia fournieri ‘10868’
Date of the original deposit: 14 Apr. 2021
Date of issue of the Receipt of the Original Deposit (domestic): 14 Jun. 2021
Date of request to convert the original deposit to a deposit under the Budapest Treaty: 2 Dec. 2022
Date of viability test by the International Depositary Authority: 23 Dec. 2022
Date of issue of the Receipt in the Case of an Original Deposit by the International Depositary Authority: 6 Feb. 2023
   FERM BP-22419: Torenia fournieri ‘DF17’
Date of the original deposit: 17 Jun. 2021
Date of issue of the Receipt of the Original Deposit (domestic): 1 Sep. 2021
Date of request to convert the original deposit to a deposit under the Budapest Treaty: 2 Dec. 2022
Date of viability test by the International Depositary Authority: 23 Dec. 2022
Date of issue of the Receipt in the Case of an Original Deposit by the International Depositary Authority: 6 Feb. 2023
   FERM BP-22420: Torenia fournieri ‘MC1’
Date of the original deposit: 17 Jun. 2021
Date of issue of the Receipt of the Original Deposit (domestic): 1 Sep. 2021
Date of request to convert the original deposit to a deposit under the Budapest Treaty: 2 Dec. 2022
Date of viability test by the International Depositary Authority: 23 Dec. 2022
Date of issue of the Receipt in the Case of an Original Deposit by the International Depositary Authority: 6 Feb. 2023
   FERM BP-22422: Torenia fournieri ‘RSF1’
Date of the original deposit: 17 Jun. 2021
Date of issue of the Receipt of the Original Deposit (domestic): 1 Sep. 2021
Date of request to convert the original deposit to a deposit under the Budapest Treaty: 2 Dec. 2022
Date of viability test by the International Depositary Authority: 23 Dec. 2022
Date of issue of the Receipt in the Case of an Original Deposit by the International Depositary Authority: 6 Feb. 2023
   FERM BP-22437: Torenia fournieri ‘11118’
Date of the original deposit: 16 Dec. 2021
Date of issue of the Receipt of the Original Deposit (domestic): 15 Feb. 2022
Date of request to convert the original deposit to a deposit under the Budapest Treaty: 2 Dec. 2022
Date of viability test by the International Depositary Authority: 23 Dec. 2022
Date of issue of the Receipt in the Case of an Original Deposit by the International Depositary Authority: 6 Feb. 2023

### (3) Characteristics of the Deposited Biological Materials

The characteristics of the deposited biological materials FERM BP-22419, FERM BP-22420, FERM BP-22437, FERM BP-22422 and FERM BP-22418 are described in the Detailed Description of the Embodiments and the Examples sections of the present disclosure. The taxonomic position and scientific characteristics that are common for these deposited biological materials are as follows:

### Taxonomic Position:

Family: Linderniaceae
Genus: Torenia
Species: Torenia fournieri Lind. ex Fourn.

### Information on Scientific Characteristics:

The genome is diploid. They are extremely tolerant to high temperature and has high environmental adaptability, being able to be cultivated under a wide range of light conditions from full sun to shade. It is hard to obtain their mutants by mutation treatment. The petals are fused at their base to form a gamopetalous flower, but the flower shape is very different from that of a normal-type Torenia flower. As described above, each strain has flower characteristics unique to the strain.

### INDUSTRIAL APPLICABILITY

The double-flowered Torenia plants (Torenia strains) produced in the present disclosure can be directly utilized as Torenia cultivars, due to their high ornamental value. They can be further utilized to produce novel cultivars at research institutes and nursery companies. Specifically, introduction of various flower colors or plant shapes of existing commercial varieties into the double-flowered Torenia plants (Torenia strains) produced in the present disclosure is expected to help produce cultivars with even higher commercial value.

### Accession Numbers

FERM BP-22419
FERM BP-22420
FERM BP-22437
FERM BP-22422
FERM BP-22418

1. A method for producing a double-flowered Torenia plant, the method comprising the steps of:
obtaining an F1 population by crossing
(Parent 1)
a Torenia plant belonging to a semi-double-flowered strain (FERM BP-22419) or
a Torenia plant that belongs to a progeny population or a mutant population of the Torenia plant belonging to the semi-double-flowered strain (FERM BP-22419) and has a flower with 7 to 10developed petals; and
(Parent 2)
a Torenia plant belonging to a multiple-carpel strain (FERM BP-22420) or
a Torenia plant that belongs to a progeny population or a mutant population of the Torenia plant belonging to the multiple-carpel strain (FERM BP-22420) and has a flower with an increased number of carpels; and
selecting, from a second- or subsequent-generation hybrid population, an individual that has a flower with 15 or more developed petals, the second- or subsequent-generation hybrid population being a population of a second or subsequent generation derived from a first-generation hybrid population, and the first-generation hybrid population being the F1 population that has been obtained.

2. A method for producing a double-flowered Torenia plant, the method comprising the steps of:
obtaining an F1 population by crossing
(Parent 3)
the double-flowered Torenia plant obtained by the method according to claim 1 or
a Torenia plant that belongs to a progeny population or a mutant population of the double-flowered Torenia plant obtained by the method according to claim 1 and that has a flower with 15 or more developed petals; and
(Parent 4)
a freely selected Torenia plant; and
selecting, from a second- or subsequent-generation hybrid population, an individual that has a flower with 15 or more developed petals and has a desired trait, the second- or subsequent-generation hybrid population being a population of a second or subsequent generation derived from a first-generation hybrid population, and the first-generation hybrid population being the F1 population that has been obtained.

3. A method for producing a double-flowered Torenia plant, the method comprising the steps of:
obtaining an F1 population by crossing
(Parent 3′)
the double-flowered Torenia plant obtained by the method according to claim 1 or
a Torenia plant that belongs to a progeny population or a mutant population of the double-flowered Torenia plant obtained by the method according to claim 1 and that has a flower with 15 or more developed petals; and
(Parent 5)
a Torenia plant belonging to a begonia-like strain (FERM BP-22422) or
a Torenia plant that belongs to a progeny population or a mutant population of the Torenia plant belonging to the begonia-like strain (FERM BP-22422) and that has a flower whose shape is bilaterally and dorsoventrally symmetric; and
selecting, from a second- or subsequent-generation hybrid population, an individual that has a flower with 15 or more developed petals arranged radially around a floral axis to form a double flower with a substantially radially symmetric corolla, the second- or subsequent-generation hybrid population being a population of a second or subsequent generation derived from a first-generation hybrid population, and the first-generation hybrid population being the F1 population that has been obtained.

4. A method for producing a double-flowered Torenia plant, the method comprising the steps of:
obtaining an F1 population by crossing
(Parent 6)
the double-flowered Torenia plant obtained by the method according to claim 3 or
a Torenia plant that belongs to a progeny population or a mutant population of the double-flowered Torenia plant obtained by the method according to claim 3 and that has a flower with 15 or more developed petals arranged radially around a floral axis to form a double flower with a substantially radially symmetric corolla; and
(Parent 7)
a freely selected Torenia plant; and
selecting, from a second- or subsequent-generation hybrid population, an individual that has a flower with 15 or more developed petals arranged radially around a floral axis to form a double flower with a substantially radially symmetric corolla and that has a desired trait, the second- or subsequent-generation hybrid population being a population of a second or subsequent generation derived from a first-generation hybrid population, and the first-generation hybrid population being the F1 population that has been obtained.

5. A method for producing a double-flowered Torenia plant, wherein the double-flowered Torenia plant obtained by the method according to claim 1 is used as one parent or both parents of a cross.

6. A double-flowered Torenia plant,
wherein the double-flowered Torenia plant is any one of the following (Plant 1), (Plant 2), or (Plant 3):
(Plant 1)
a Torenia plant belonging to a fully double-flowered strain (FERM BP-22437) or a carnation-like fully double-flowered strain (FERM BP-22418),
(Plant 2)
a Torenia plant that belongs to a progeny population of the Torenia plant described in (Plant 1) and that has a flower with 15 or more developed petals; or
(Plant 3)
a Torenia plant that belongs to a mutant population of the Torenia plant described in (Plant 1) or (Plant 2) and that has a flower with 15 or more developed petals.

7. A double-flowered Torenia plant,
wherein the double-flowered Torenia plant is any one of the following (Plant 1′), (Plant 2′), or (Plant 3′):
(Plant 1′)
a Torenia plant belonging to a carnation-like fully double-flowered strain (FERM BP-22418),
(Plant 2′)
a Torenia plant that belongs to a progeny population of the Torenia plant described in (Plant 1′) and that has a flower with 15 or more developed petals arranged radially around a floral axis to form a double flower with a substantially radially symmetric corolla; or
(Plant 3′)
a Torenia plant that belongs to a mutant population of the Torenia plant described in (Plant 1′) or (Plant 2′) and that has a flower with 15 or more developed petals arranged radially around a floral axis to form a double flower with a substantially radially symmetric corolla.

8. A method for producing a double-flowered Torenia plant,
wherein the double-flowered Torenia plant according to claim 6 is used as one parent or both parents of a cross.

9. A plant body of the double-flowered Torenia plant according to claim 6.

10. A method for cultivating a double-flowered Torenia plant, a method for producing a plant body of a double-flowered Torenia plant, or a method for producing a strain of a double-flowered Torenia plant,
wherein the double-flowered Torenia plant according to claim 6 is used.

11. A method for producing a double-flowered Torenia plant,
wherein the double-flowered Torenia plant according to claim 7 is used as one parent or both parents of a cross.

12. A plant body of the double-flowered Torenia plant according to claim 7.

13. A method for cultivating a double-flowered Torenia plant, a method for producing a plant body of a double-flowered Torenia plant, or a method for producing a strain of a double-flowered Torenia plant,
wherein the double-flowered Torenia plant according to claim 7 is used.

14. A method for producing a double-flowered Torenia plant, wherein the double-flowered Torenia plant obtained by the method according to claim 2 is used as one parent or both parents of a cross.

15. A method for producing a double-flowered Torenia plant, wherein the double-flowered Torenia plant obtained by the method according to claim 3 is used as one parent or both parents of a cross.

16. A method for producing a double-flowered Torenia plant, wherein the double-flowered Torenia plant obtained by the method according to claim 4 is used as one parent or both parents of a cross.
